(19) United States
(12) Plant Patent Application Publication
(10) Pub. No.: US 2014/0020142 P1
(43) Pub. Date: Jan. 16, 2014

(54) Avocado tree named 'Eugenin'

(50) ;

(71) Applicant: Javier Eugenin, Hijuelas (CL)

(72) Inventor: Javier Eugenin, Hijuelas (CL)

(21) Appl. No.: 13/507,592

(22) Filed: Jul. 12, 2012

(51) Int. Cl. A01H 5/00

(52) U.S. Cl. PLT/200

(57) ABSTRACT

The ‘Eugenin’ avocado variety is notable for its larger fruit size than that of the ‘Hass’ variety.

### LATIN NAME OF THE GENUS AND SPECIES

Persea americana Mill.

### VARIETAL DENOMINATION

‘Eugenin’

### BACKGROUND OF THE INVENTION

‘Eugenin’ is a new and distinct variety of avocado tree Persea americana Mill. The ‘Eugenin’ variety was first discovered in 1997 by Mr. Javier Eugenin, who noted a tree in his cultivated avocado orchard located in Hijuelas, in the Aconcagua Valley in Chile, more particularly the orchard called Parcela Magallanes. The tree was discovered after observing through several years that its fruit size was consistently higher than the ‘Hass’ trees from the orchard. After several evaluations, it was determined that the ‘Eugenin’ tree consistently produced larger fruit than ‘Hass’ trees.

### SUMMARY OF THE INVENTION

The ‘Eugenin’ discovered tree is presumed to be a mutated bud (it is a whole tree mutation) from a spontaneous mutation of ‘Hass’. Budwood from the originally discovered ‘Eugenin’ mother tree was grafted into new trees, which were planted in January 2000, as a non-propagation trial in the orchard La Escondida in Nogales, Chile. It has since been observed to remain true to type and the material and mutation is stable. The tree was discovered, after observing through several years, that the fruit size was consistently larger than corresponding ‘Hass’ trees.

### BRIEF DESCRIPTION OF THE PHOTOGRAPHS

The accompanying photographs show specimens of the tree and plant parts of the new ‘Eugenin’ variety.

FIG. 1 is a photograph of the originally discovered ‘Eugenin’ mother tree;

FIG. 2 is a photograph depicting ‘Eugenin’ trees flowering;

FIG. 3 is a photograph depicting fruit and leaves of ‘Eugenin’;

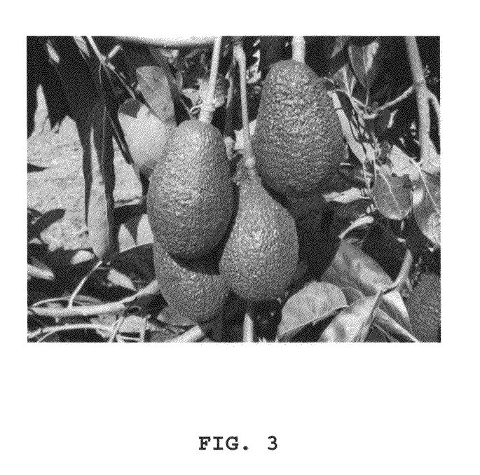

FIG. 4 is a photograph depicting fruit and leaves next to a hand holding a pen for size reference; and

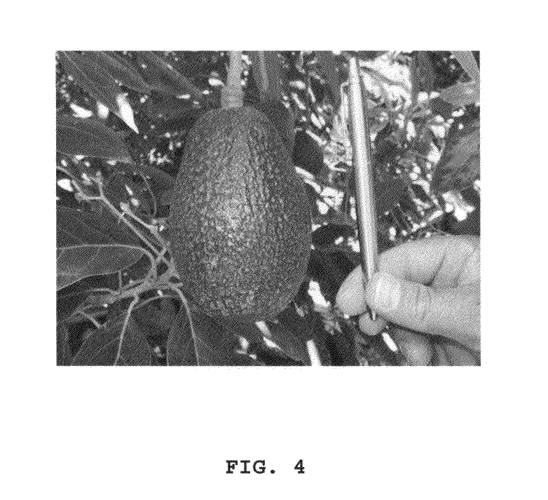

FIG. 5 is a photograph depicting sectioned fruit of the ‘Eugenin’ above ‘Hass’ fruit, illustrating that the fruit and seed is larger in ‘Eugenin’ than that of ‘Hass’.

### DETAILED BOTANICAL DESCRIPTION OF THE VARIETY

The following is a detailed botanical description of the ‘Eugenin’ variety, sometimes referred to by the code names Andes 4 or GAMA 4021 by the breeder.

The following detailed botanical description is based on observations of trees which were grafted budwood from the ‘Eugenin’ mother tree and planted in January 2000, as a non-propagation trial in the orchard “La Escondida” in Nogales, Chile. The 2007 report is based on a four-year study of these trees planted in 2000, ensuring that the material and mutation is stable. The ‘Eugenin’ variety is nearly identical to ‘Hass’, except that it has a slight early flowering as compared to ‘Hass’ and produces larger fruit than ‘Hass’. Otherwise, it is nearly identical in color and appearance as to ‘Hass’ trees.

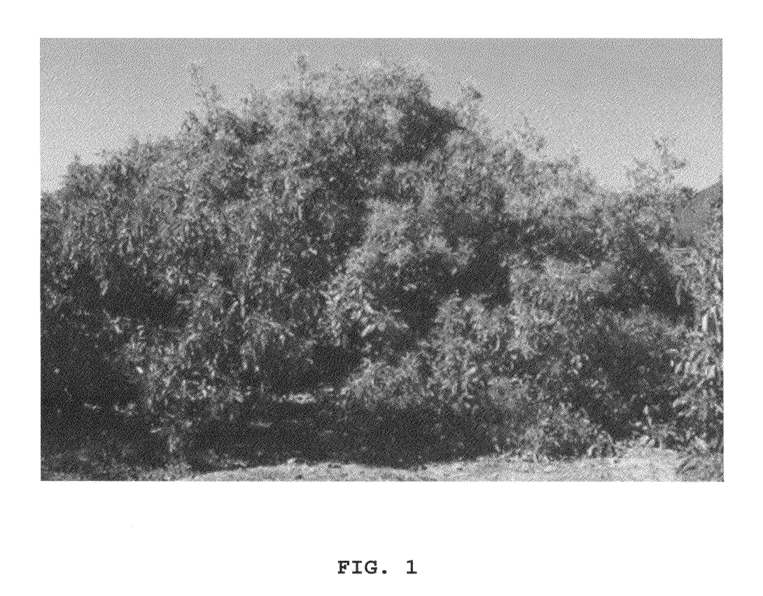

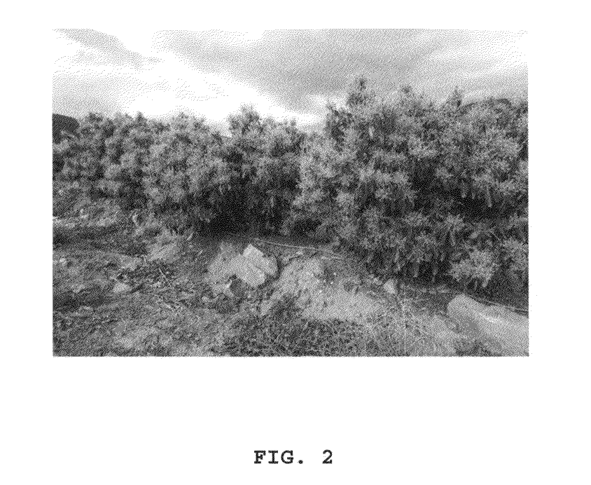

The ‘Eugenin’ tree presents a spreading growth habit, as illustrated in FIGS. 1 and 2. The young shoots are reddish in color. The anise aroma of the leaf blade is weak or absent. The inflorescence flowering type is similar to ‘Hass’ Type A.

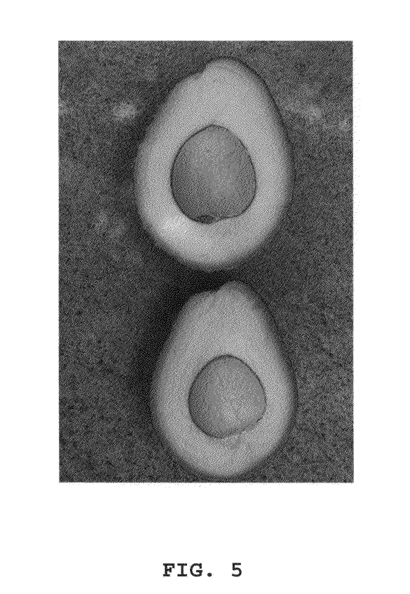

With respect to the fruit, the surface of the mature fruit is rough, similar to ‘Hass’. The thickness of the pedicel compared to the peduncle, at the junction, is thicker. The pedicel length is long, similar to ‘Hass’. However, the pedicel shape is cylindrical, and does not include a “nail head”. The ripe fruit color is dark purple or black, similar to ‘Hass’. The thickness of the ripe fruit skin is moderately thick, as ‘Hass’. As illustrated in FIG. 5, the fruit is larger than that of ‘Hass’. Moreover, the polar and equatorial diameter of the ‘Eugenin’ fruit is greater than ‘Hass’, resulting in larger fruit and higher average weight. Furthermore, the polar diameter/equatorial diameter ratio is greater, so that the ‘Eugenin’ fruit is more elongated than ‘Hass’. The shape of the seed and longitudinal section is ovate. The seed size is generally larger, as illustrated in FIG. 5.

The ‘Eugenin’ variety has a slight early flowering as compared to ‘Hass’. The ‘Eugenin’ variety flowering tends to be higher than ‘Hass’, and reaches a peak approximately a week before ‘Hass’. The end of the flowering period for ‘Eugenin’ is approximately a week before ‘Hass’.

The time of fruit maturity for harvesting is late, similar to ‘Hass’, in that the ‘Eugenin’ variety reaches or exceeds the minimum harvest maturity (23% dry matter) almost at the same time as that of ‘Hass’.

What is claimed is:

1. A new and distinct variety of avocado tree, substantially as illustrated and described herein.
